(19) United States
(12) Patent Application Publication
(10) Pub. No.: US 2025/0011787 A1
(43) Pub. Date: Jan. 9, 2025

(54) Retroelement-generated transcription factor decoys

(71) Applicants: THE J. DAVID GLADSTONE INSTITUTES, A TESTAMENTARY TRUST ESTABLISHED UNDER THE WILL OF J. DAVID GLADS, San Francisco, CA (US); REGENTS OF THE UNIVERSITY OF CALIFORNIA, Oakland, CA (US)

(72) Inventors: Seth Shipman, San Francisco, CA (US); Katherine Crawford, Oakland, CA (US)

(21) Appl. No.: 18/703,267

(22) PCT Filed: Oct. 21, 2022
(86) PCT No.: PCT/US2022/078485

§ 371 Date: Apr. 19, 2024

Related U.S. Application Data
(60) Provisional application No. 63/270,304, filed on Oct. 21, 2021.

(51) Int. Cl. C12N 15/113

(52) U.S. Cl. CPC C12N 15/1135; C12N 2310/13

(57) ABSTRACT

Described herein are retrons that are engineered to have binding sites for transcription factors, as well as compositions, systems, and methods for obtaining and using such engineered retrons.

### CROSS-REFERENCE TO RELATED APPLICATIONS

This application claims the benefit of priority to U.S. Provisional Ser. No. 63/270,304, filed Oct. 21, 2021, which is incorporated by reference as if fully set forth herein.

Incorporation by Reference of Sequence Listing Provided as an XML File A Sequence Listing is provided herewith as an xml file, “2280006.xml” created on Oct. 20, 2022 and having a size of 45,056 bytes. The content of the xml file is incorporated by reference herein in its entirety.

### BACKGROUND

Transcription factors orchestrate the most dynamic processes in cells—from producing cascades of antiviral and antibiotic resistance proteins to differentiation and metastasis. They do this by binding short sequences of DNA to exert their effect on gene expression. A change in the abundance of even a single transcription factor can induce a pattern of gene expression that fundamentally alters the transcriptome and physiology of a cell. The effect that a transcription factor has on gene expression is a function of both the abundance of the factor itself as well as the abundance of its binding site in DNA. As the number of DNA binding sites increases, the effect of each transcription factor molecule decreases. Yet, in cells, protein abundance changes and the number of DNA binding sites is static, so the transcription factor amount is the only point of dynamic control.

Current attempts at using oligonucleotides as ‘decoys’ to bind and modulate the function of transcription factors have led to several problems: (1) decoy oligonucleotides have a time-limited effect because they are quickly degraded by endogenous nucleases; (2) they cannot be produced on demand to enable temporal control; and (3) they cannot be delivered with cell-type selectivity or produced under the control of other cellular pathways.

### SUMMARY

Currently, oligonucleotide transcription factor decoys are small pieces of synthesized DNA that contain a transcription factor binding site. These small pieces of DNA need to be exogenously administered, delivered to the cells of interest at a low frequency, and are degraded quickly. In contrast, engineered retrons can be made by using a reverse transcriptase system to create transcription factor decoys that can be provided continuously, or by inducible production in the cells of interest, thus eliminating the lifetime and delivery problems of the current technologies.

Described herein are engineered retrons that include a transcription factor binding site within the retron msd region. Examples of such transcription factor binding site include site that bind a STAT1, STAT3, STAT5a, NF-κB, E2F, GATA-1, GATA-2, GATA-3, STAT-1, STAT-6, Ets, AP-1, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, OCT4, NANOG, SOX2, TBX5, NKX2-5, Forkhead, ETS, ELF3, ELF5, EHF, SPDEF, JunB, FOSL2, ATF2, ATF7, PBX1, C/EBPb, C/EBPs, VDR, PPARy, RXR, RUNX1, RUNX2, FLI1, TAL1/SCL, FOG1, GFI1b, EKLF, GABPa, LYL1, LMO2, MEIS1, PU.1, ERG, Sp1, Sp2, Sp3, Sp4, Myc, FOXO-1, FOXO-23, FOXO-4, FOXO-6, TCF21, MZF1-A, MLL-AF9, CBFb-SMMHC, GABP, SIX1, PML-RARa, CBFb, and p53 transcription factor, or a combination of such transcription factors.

The retrons to be engineered can be from any prokaryotic source. Almost 50,000 retrons have been bioinformatically identified and any them can be engineered as described herein. See, e.g., website at pubmed.ncbi.nlm.nih.gov/33275130/. In many cases, the retrons to be engineered can be from bacteria. For example, the retrons to be modified include modified Escherichia coli retrons (e.g., Ec67, Ec73, EC83, EC86, EC107), myxobacteria retrons (e.g., Mx65, Mx162), Salmonella enterica retrons (e.g., St85, Se72), Vibrio cholerae retrons (e.g., Vc81, Vc95, Vc137), Stigmatella aurantica retrons (e.g., Sa163), Nannocystis exedens retrons (Ne160, Ne144), Vibrio parahaemolyticus retrons (e.g., Vp96), Proteus mirabilis retrons (e.g., Pmi1), Klebsiella pneumoniae retrons (e.g., Kpn1), Flexibacter elegans retrons (e.g., Fei1), Corallococcus coralloides retrons (e.g., Coo1), Cystobacter ferrugineus retrons (e.g., Cfe1), Melittangium lichenicola retrons (e.g., Mli1), Chondromyces apiculatus retrons (e.g., Cap1), Sorangium cellulosum retrons (e.g., Sce1) or combinations thereof. In some cases, the engineered retrons are modified Eco1 or Eco2 retrons.

Expression systems encoding the engineered retrons as well as modified cells that include the engineered retrons or the expression systems are also described herein.

The engineered retrons, expression systems, and cells can be administered to a subject. The subject can be suffering from (is suspected of having or is prone to developing) a disease or condition that can be treated by reducing the function of the transcription factor that binds to the binding site of the engineered retrons.

### DESCRIPTION OF THE FIGURES

FIG. 1A-1D describe the retron system and show that it produces single-stranded DNA (ssDNA). FIG. 1A shows the endogenous form of the retron, consisting of a reverse transcriptase, a ncRNA (pink), which is reverse transcribed into reverse transcribed DNAs (RT-DNA, blue). The complementary RNA is then degraded by native host factors, such as RNase H. The endogenous retron also contains an accessory protein, which is not necessary for RT-DNA production, as shown in FIG. 1B, where the RT and ncRNA are sufficient to produce a specific piece of ssDNA in E. coli. FIG. 1C-1D describe a method and result for measuring the quantity of RT-DNA using qPCR, with two primers that anneal to the RT-DNA (light blue and black) and two primers that anneal to the production plasmid (pink and black). Using the delta-delta Ct method, we calculate a 10× fold-change in the amount of RT-DNA upon induction of the RT.

FIG. 2A-2C show that the retron system can be used to generate DNA in cells via a reverse transcriptase to generate many copies of decoy binding sites for transcription factors. FIG. 2A is a schematic of a retron operon that encodes an ncRNA and reverse transcriptase, where the reverse transcriptase can synthesize many DNA copies of the msd portion of the ncRNA to generate multiple single-stranded reverse transcribed DNAs. As illustrated herein, these RT-DNAs can be decoys that bind transcription factors, which dilutes or reduces the function of the transcription factor. FIG. 2B is a schematic of a transcription factor binding to its natural genomic site and thereby regulate transcription of an operably linked gene. FIG. 2C is a schematic of multiple decoy (RT-DNA) retrons that each contain a binding motif for a transcription factor. Transcription factors bound to such retron decoys are not available to bind to genomic sites that they would normally regulate.

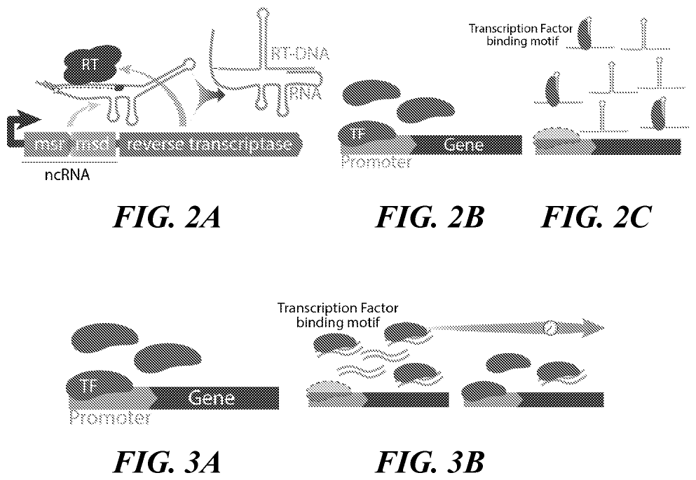

FIG. 3A-3B illustrates the problems of oligonucleotide decoys. FIG. 3A is a schematic of a transcription factor binding to its natural genomic site and thereby regulate transcription of an operably linked gene. FIG. 3B is a schematic of a transcription factor in the presence of one of its genomic binding sites with decoy oligonucleotides that have a binding motif for the transcription factors. Although, such oligonucleotides can be used to attenuate transcription factor binding to promoters in the genome, the oligonucleotide decoys degrade over time, may not be present in sufficient amounts, are not easily regulated or targeted to appropriate cells, and have limited delivery options.

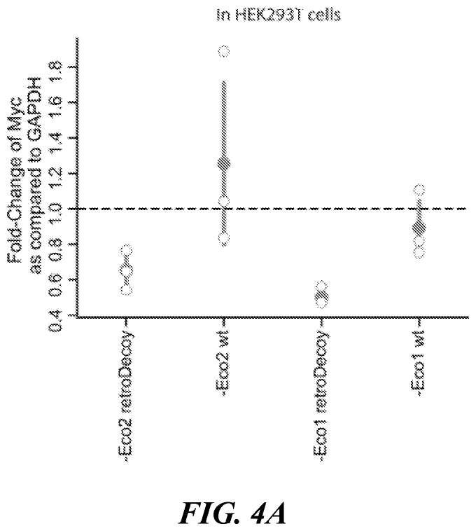

FIG. 4A-4B illustrate that retron decoys can down-regulate transcription driven by the STAT3 transcription factor, which can induce transcription of Myc. FIG. 4A graphically illustrates that inducing expression and reverse transcription of retron-Eco1 and -Eco2 RT-DNA retron decoys containing the STAT3 motif results in downregulation of Myc transcription, whereas wild-type retrons-Eco1 and -Eco2 do not. FIG. 4B graphically illustrates that ligated synthetic decoy oligonucleotides downregulate Myc in squamous cell carcinomas Cal33 cells but a control mutant oligonucleotide does not.

### DETAILED DESCRIPTION

Described herein are retrons that include binding sites for transcription factors, referred to as retron decoys. Such retron decoys can reduce the activity of transcription factors and reduce negative effects of the dynamic processes induced by transcription factors.

The ssDNA generated by the retron reverse transcriptase forms a double-stranded DNA region to which the transcription factors bind. Although double-stranded DNA oligonucleotides can be introduced into a cell, they cannot be introduced into cells in large amounts because double-stranded DNA oligonucleotides cannot be synthesized in large amounts in vivo. The synthesis of retrons can also be controlled, not just at the transcriptional level, but also at the reverse transcriptional level. Larger amounts of retrons can be made than by simply inducing transcription of an RNA because the retron reverse transcriptase can make many copies of the retron DNA from a single retron RNA or from many RNA copies.

Applicants believe that retrons have not previously been used as transcription factor decoys. However, retrons have been used for genome engineering in two contexts: in bacteria with the λ Red Beta recombinase for recombineering (Farzadfard et al. Science 346, 1256272, (2014)); and in eukaryotes, as a homology-directed repair (HDR) template for Cas9 editing (Sharon et al. Cell 175, 544-557.e516, (2018)) in yeast. Despite tremendous promise, these applications suffered from lower-than-expected efficiency and context-restriction, which may stem from elements in the endogenous form of the retron. These include (1) a branched structure with a phosphodiester bond linking the 5′ end of the ssDNA to a 2′ hydroxyl of the msr RNA, (2) invariant flanking regions that may be required for retron reverse transcription, but are not part of the repair template, (3) limited total length, and (4) a native poly T stretch that functions as a terminator for Pol III transcription; however, the polyT stretch is generally changed (shortened and/or removed) so as to not stop transcription at that point in the retron.

The engineered retrons described herein do not have such problems because the inventors have identified the portion of the retron where additional sequences can be added without reducing synthesis of the retron. The region of the retron where added sequences can be inserted is the region that forms double stranded DNA, which is the very region where the transcription factor binding sites are best placed. Such inserted sequences do not interfere with the flanking sequences needed for reverse transcription or with a poly T tail used for termination.

### Engineered Retrons

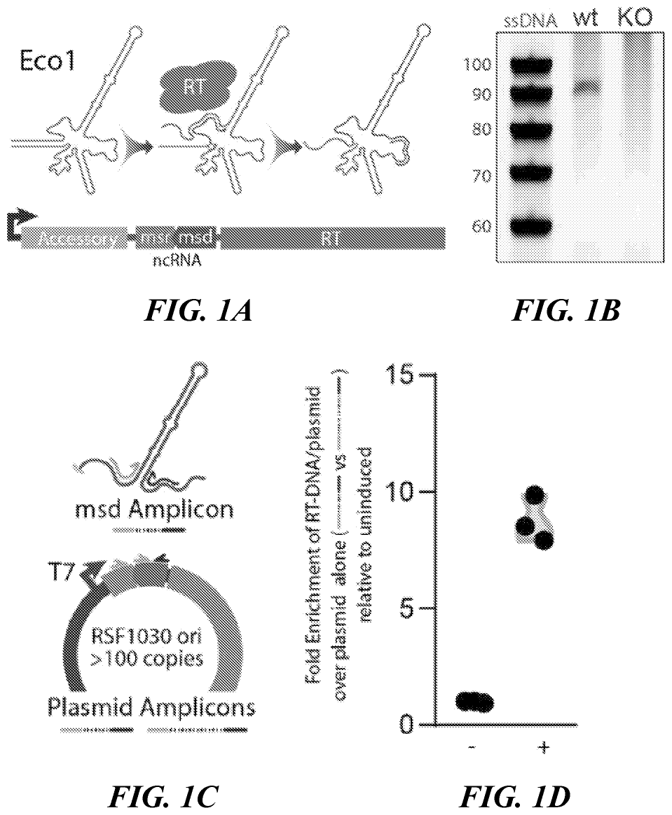

The present disclosure provides an engineered retron that is modified to include transcription factor binding sites. A typical retron operon consists of a reverse transcriptase (RT), a non-coding RNA (ncRNA) that is both the primer and template for the reverse transcriptase, and one or more accessory proteins (FIG. 1A). The reverse transcriptase partially reverse transcribes the ncRNA to produce a single stranded RT-DNA with a characteristic hairpin structure, which in wild type retrons varies in length from about 48 to 163 bases. The ncRNA can be sub-divided into a region that is reverse transcribed (msd) and a region that remains RNA in the final molecule (msr); these regions are partially overlapping.

One of the first described retrons found in E. coli is called Eco1 (previously called ec86). In BL21 E. coli cells, this retron is present and active, producing reverse transcriptase DNA that can be detected at the population level. The wild type Eco1 retron can be eliminated from BL21 E. coli cells by removing the retron operon from the genome (FIG. 1i). In the absence of this native operon, the ncRNA and reverse transcriptase can be expressed from a plasmid lacking the accessory protein. Since the accessory protein is a core component of the phage-defense conferred by retrons, this reduced system would reduce phage defense capacity, yet cells with ncRNA-reverse transcriptase encoding plasmids continue to produce abundant reverse transcribed DNA.

The inventors quantified the reverse transcribed DNA from Eco1 retrons using a relative qPCR assay that compared amplification by primers that bind the msd region. Such msd-primers can use both the reverse transcribed-DNA and the ncRNA-reverse transcriptase encoding plasmids as a template (blue-black primers shown in FIG. 1C). A second set of primers was used to amplify only a portion of the plasmid (red-black primers shown in FIG. 1C). In E. coli lacking an endogenous retron, overexpression of the ncRNA and RT from a plasmid yielded an approximate 8-10 fold enrichment of the reverse transcribed DNA/plasmid region over the plasmid alone, which is evidence of substantial reverse transcription (FIG. 1D).

In retrons engineered to include transcription factor binding sites, the post-msd sequence is, for example, modified within its self-complementary region (which has sequence complementarity to the pre-msr sequence). Hence, the length of the self-complementary region is increased relative to the corresponding region of a native retron. Such modifications result in an engineered retron that can provide enhanced production of msDNA that includes one or more transcription factor binding sites.

In certain embodiments, the retron complementary region has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from 1 to 50 nucleotides longer than the native or wild-type complementary region, including any length within this range, such as 1, 2, 3,4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides longer. In certain embodiments, the self-complementary region has a length ranging from 1 to 16 nucleotides longer than the wild-type complementary region. The single-stranded DNA generated by the engineered retron can form the double-stranded region be used in various applications.

In certain embodiments, the msr gene, msd gene, and ret gene are derived from bacterial retrons including, without limitation, modified Escherichia coli retrons (e.g., Ec67, Ec73, EC83, EC86, EC107), myxobacteria retrons (e.g., Mx65, Mx162), Salmonella enterica retrons (e.g., St85, Se72), Vibrio cholerae retrons (e.g., Vc81, Vc95, Vc137), Stigmatella aurantica retrons (e.g., Sa163), Nannocystis exedens retrons (Ne160, Ne144), Vibrio parahaemolyticus retrons (e.g., Vp96), Proteus mirabilis retrons (e.g., Pmi1), Klebsiella pneumoniae retrons (e.g., Kpn1), Flexibacter elegans retron (e.g., Fei1), Corallococcus coralloides retrons (e.g., Coo1), Cystobacter ferrugineus retrons (e.g., Cfe1), Melittangium lichenicola retron (e.g., Mli1), Chondromyces apiculatus retrons (e.g., Cap1), Sorangium cellulosum retrons (e.g., Sce1) or combinations thereof. In some cases, the retron decoys can be generated using modified ncRNAs from two retrons, Eco1 and Eco2.

An Eco1 wild type retron non-coding RNA (ncRNA) sequence is shown below as SEQ ID NO: 1.

| | |
|---|---|
| 1 | ATGCGCACCC TTAGCGAGAG GTTTATCATT AAGGTCAACC |
| 41 | TCTGGATGTT GTTTCGGCAT CCTGCATTGA ATCTGAGTTA |
| 81 | CTGTCTGTTT TCCTTGTTGG AACGGAGAGC ATCGCCTGAT |
| 121 | GCTCTCCGAG CCAACCAGGA AACCCGTTAT TTCTGACGTA |
| 161 | AGGGTGCGCA |

An example of an Eco1 human-codon optimized reverse transcriptase (RT) sequence that can be used is shown below as SEQ ID NO: 2.

| | |
|---|---|
| 1 | ATGAAATCTG CAGAGTATCT GAATACGTTC CGCCTTAGGA |
| 41 | ATTTGGGCCT CCCCGTGATG AACAATCTCC ACGATATGAG |
| 81 | CAAGGCGACT CGAATATCCG TGGAAACGCT GAGACTGCTC |
| 121 | ATCTATACAG CAGACTTTCG GTACAGGATC TACACGGTCG |
| 161 | AAAAGAAGGG GCCTGAGAAA CGCATGCGAA CAATTTATCA |
| 201 | ACCTAGCCGA GAGCTCAAGG CGTTGCAGGG CTGGGTTCTT |
| 241 | CGAAACATCC TTGACAAACT CTCATCATCA CCCTTTAGTA |
| 281 | TTGGGTTTGA AAAGCACCAA AGCATCCTTA ACAACGCGAC |
| 321 | GCCACACATA GGTGCCAATT TCATATTGAA CATCGACTTG |
| 361 | GAGGATTTTT TTCCGAGCCT CACAGCCAAT AAAGTGTTCG |
| 401 | GTGTTTTTCA CAGTCTTGGG TACAATCGCC TTATTAGTTC |
| 411 | CGTTCTTACC AAGATTTGTT GTTACAAGAA TCTCTTGCCC |
| 481 | CAGGGAGCAC CCAGCAGTCC GAAATTGGCG AATTTGATTT |
| 521 | GTTCCAAGCT CGATTATCGA ATACAAGGGT ACGCGGGCAG |
| 561 | CCGGGGACTC ATCTATACCC GCTACGCAGA CGATCTTACG |
| 601 | CTGTCTGCCC AATCAATGAA GAAGGTCGTA AAGGCGCGGG |
| 641 | ATTTCTTGTT TTCTATCATC CCGTCCGAGG GCTTGGTAAT |
| 681 | TAATTCCAAA AAGACTTGTA TCTCAGGACC ACGATCTCAG |
| 721 | CGAAAAGTGA CAGGACTCGT CATTTCTCAA GAAAAAGTCG |
| 761 | GTATAGGGAG AGAGAAGTAT AAGGAAATCC GCGCGAAGAT |
| 801 | CCACCACATA TTCTGTGGCA AGAGCAGCGA GATAGAACAC |
| 841 | GTCCGAGGCT GGTTGTCCTT CATACTGAGC GTGGACTCAA |
| 881 | AAAGCCACCG CCGGTTGATC ACCTATATTT CAAAACTGGA |
| 921 | AAAGAAATAT GGAAAGAACC CACTCAACAA AGCTAAAACA |
| 961 | TAG |

The Eco2 human-codon optimized reverse transcriptase (RT) sequence is shown below as SEQ ID NO: 3.

| | |
|---|---|
| 1 | ATGACAAAAA CTTCAAAGCT GGATGCGCTG CGGGCGGCTA |
| 41 | CTAGTAGGGA AGATTTGGCG AAGATTCTCG ACATAAAGTT |
| 81 | GGTGTTTCTG ACAAACGTGT TGTACCGCAT AGGATCCGAC |
| 121 | AACCAGTATA CGCAATTCAC AATACCCAAA AAGGGTAAAG |
| 161 | GTGTCCGCAC CATCAGCGCA CCAACGGACC GACTTAAGGA |
| 201 | TATACAGAGG AGGATTTGTG ATCTTCTTAG TGACTGTAGG |
| 241 | GATGAAATCT TTGCGATTAG GAAGATCTCT AATAATTACT |
| 281 | CATTCGGCTT CGAAAGAGGA AAATCAATTA TACTCAATGC |
| 321 | TTACAAGCAT CGAGGGAAGC AAATTATATT GAACATCGAC |
| 361 | CTTAAGGACT TCTTTGAGAG CTTTAACTTT GGGAGAGTCC |
| 401 | GGGGGTACTT TCTCTCCAAC CAGGACTTCT TGTTGAACCC |
| 441 | AGTIGTGGCA ACAACGTTGG CGAAGGCCGC CTGCTACAAC |
| 481 | GGGACTCTGC CTCAGGGGTC CCCATGTTCC CCTATTATAA |
| 521 | GTAACCTTAT CTGTAACATT ATGGACATGC GGCTCGCAAA |
| 561 | GCTCGCCAAG AAGTACGGCT GCACTTATAG TCGATATGCG |
| 601 | GATGACATTA CGATCAGCAC CAATAAAAAT ACCTTCCCGT |
| 641 | TGGAGATGGC GACTGTGCAG CCTGAAGGGG TTGTGCTGGG |
| 681 | CAAAGTGCTC GTAAAGGAGA TTGAAAATTC AGGTTTCGAG |
| 721 | ATTAACGATT CTAAGACTAG ATTGACCTAC AAAACAAGTA |
| 761 | GGCAAGAAGT CACCGGGCTG ACGGTTAATC GGATTGTAAA |
| 801 | CATTGATCGG TGCTACTACA AAAAGACGAG GGCGCTGGCT |
| 841 | CACGCATTGT ATCGGACAGG AGAATATAAG GTCCCAGACG |
| 881 | AGAACGGTGT TCTGGTATCT GGAGGGCTTG ACAAGTTGGA |
| 921 | GGGTATGTTT GGGTTTATCG ACCAGGTGGA TAAATTCAAC |
| 961 | AACATTAAAA AAAAGTTGAA TAAGCAACCC GACAGATATG |
| 1001 | TTCTGACAAA TGCCACTTTG CACGGATTTA AGCTCAAATT |
| 1041 | GAACGCCAGG GAGAAAGCCT ATAGCAAATT CATCTACTAC |
| 1081 | AAATTCTTCC ACGGTAATAC TTGTCCCACG ATCATAACAG |
| 1121 | AGGGTAAGAC GGATAGGATT TACCTTAAAG CTGCCCTCCA |
| 1161 | TAGCCTCGAG ACAAGTTATC CTGAACTGTT TCGGGAGAAA |
| 1201 | ACAGATAGTA AGAAGAAGGA GATAAATCTG AATATTTTTA |
| 1241 | AAAGCAATGA GAAGACCAAG TATTTCCTGG ATCTCAGCGG |
| 1281 | CGGCACAGCA GACCTCAAGA AATTCGTGGA ACGCTACAAA |
| 1321 | AATAACTACG CTTCCTATTA CGGCAGCGTA CCGAAACAAC |
| 1361 | CGGTGATAAT GGTGCTTGAT AACGACACAG GCCCGTCAGA |
| 1401 | CCTGTTGAAC TTTTTGAGAA ACAAAGTTAA GAGTTGTCCA |
| 1441 | GATGATGTAA CAGAAATGCG CAAGATGAAG TACATACATG |
| 1481 | TGTTTTACAA TCTGTACATA GTTCTGACTC CCCTGTCTCC |
| 1521 | ATCTGGAGAG CAAACGTCTA TGGAGGACCT CTTTCCTAAA |
| 1561 | GATATATTGG ACATTAAGAT AGATGGCAAG AAATTCAATA |
| 1601 | AAAACAATGA CGGTGACTCC AAAACAGAGT ATGGGAAGCA |
| 1001 | CATATTCTCA ATGCGCGTTG TACGAGATAA AAAGAGGAAG |
| 1001 | ATAGATTTCA AGGCATTTTG CTGTATCTTC GATGCTATTA |
| 1001 | AGGATATTAA AGAACATTAC AAACTGATGT TGAATTCCTA |
| 1001 | G |

An Eco2 wild-type retron non-coding RNA (ncRNA) sequence is shown below as SEQ ID NO: 4.

| | |
|---|---|
| 1 | CACGCATGTA GGCAGATTTG TTGGTTGTGA ATCGCAACCA |
| 41 | GTGGCCTTAA TGGCAGGAGG AATCGCCTCC CTAAAATCCT |
| 81 | TGATTCAGAG CTATACGGCA GGTGTGCTGT GCGAAGGAGT |
| 121 | GCCTGCATGC GT |

Variants can occur amongst natural populations of retrons. The retrons can also have sequence variations that do not negatively affect their synthesis. Such variants and retrons with sequence variations can also be used as retron decoys. For example, any of the nucleotide sequences described herein can have at least about 80-100% sequence identity to the sequences described herein, including any percent identity within this range, such as at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence described herein.

### Transcription Factor Binding Sites

The retron decoys that are synthesized in vivo can include a retron msd DNA that has a transcription factor binding site within the double-stranded region of the msd DNA. Any transcription binding site can be included in such retron msd DNAs. Examples of transcription factor binding sites include binding sites for the AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21, or combinations of such transcription factors.

For example, a binding site for the STAT3 transcription factor can have the following sequence: CATTTCCCGTAAATC (SEQ ID NO: 5).

In another example, a binding site for a NF-κB transcription factor can have the following sequence: CCTTGAGGGGATTTCCCCCC (SEQ ID NO: 6).

In another example, a glucocorticoid receptor responsive element can have the sequence GGTACANNNTGT(T/C)CT (SEQ ID NO: 7), wherein each N is independently a T, C, G or A and wherein (T/C) is either a T or a C nucleotide.

In another example, KLF6, KLF9 and/or KLF15 binding sites can include the following sequence: GATCCTTTGCCTCCTTCGATCCTTTGCCTCCTTCAAG (SEQ ID NO: 8) or GGTGTTTGGGAGAGCTTTGGGAGGATACG (SEQ ID NO: 9). Use of such binding sites as decoys can inhibit neuropathic and neuro-inflammatory pain (see US 20170247694).

Additional examples of transcription factor binding sites are shown in Table 1.

TABLE 1
Examples of Transcription Factors and their Binding Sites

| Transcription | |
|---|---|
| factor | Binding motif |
| OCT4 | TTT(T/G)(C/G)(A/T)T(A/T) |
| SOX2 | CTTTGTC |
| NANOG | TAAT[GT][GT] or [CG][GA][CG]C[GC]ATTAN[GC] |
| | (SEQ ID NO: 10) |
| TBX5 | CTGTCA or AGGTGTGA |
| NKX2-5 | TCTTCTCACACCTTTGAAGTGG (SEQ ID NO: 11) |
| Forkhead/Ets | CATAACAGGAA (SEQ ID NO: 12) |
| C/EBP family: | TTGTGAAA |
| ATF family: | TGACGTCA |
| AP-1 family | TGAGTCAC |
| (JunB): | |
| FOSL2 | TGAGTCAC |
| Sp family: | GGGGCGGGGCG (SEQ ID NO: 13) or |
| | GGGCGGTCCCGGGCGG (SEQ ID NO: 14) |
| KLF4 | GGGGCGGGGCG (SEQ ID NO: 15) |
| KLF5 | GGGGCGGGGCG (SEQ ID NO: 16) |
| STAT1 | CTTCCTAGAAA (SEQ ID NO: 17) |
| GR | AGAACAGAATGTTCT (SEQ ID NO: 18) |
| Erg/Fli1 | ACAGGAAGATNN (SEQ ID NO: 19) |
| GATA-2 | NCAGATAAGA |
| Tal1/Scl/Lyl1 | TGCAGCTGCTG (SEQ ID NO: 21) |
| RUNX1 | CTGTGGTT |
| GATA family: | NCNCNCCCN or |
| | (T/A)GATA(A/G) |
| GFI1b | AAATCACAGC (SEQ ID NO: 39) |
| ELK1 | CCGGAAGT |
| GABPa | ACCGGAG |
| E2A | CANNTG |
| MEIS2 | TGACAG or TGATTTATG |
| Myc: | CACGTG |
| FOXO family: | GTAAA(C/T)A |
| TCF21: | NAACAGCTGG |
| MZF1-A | AGTGGGGA or |
| | CGGGnGAGGGGGAA (SEQ ID NO: 24) |
| p53 | 5'-PuPuPuC(A/T)(T/A)GPyPyPy-3' |
| MLL CXXC | CCCTGCGCAGGG (SEQ ID NO: 40) |
| GABP | ACCGGAGNN or CACTTCCTGT (SEQ ID NO: 41) |
| SIX1 | GTAACCTGANN |
| PML-RARa | GAGGTCAAAAGGTCAATG (SEQ ID NO: 42) or |
| | (A/G)G(G/T)TCANNNNNAGGTCA (SEQ ID NO: 43) |

Such transcription factor binding site sequences can be included in the double-stranded region of msd DNA, which is encoded by the retron ncRNA.

For example, an Eco1 STAT3 Retron Decoy ncRNA sequence that includes a STAT3 binding site is shown below as SEQ ID NO: 25, where the STAT3 binding site is highlighted in bold with underlining.

| | |
|---|---|
| 1 | ATGCGCACCC TTAGCGAGAG GTTTATCATT AAGGTCAACC |
| 41 | TCTGGATGTT GTTTCGGCAT CCTGCATTGA ATCTGAGTTA |
| 81 | CTGTCTGTTT TCCTTGTTGG AACATTTCCC GTAAATCGCC |
| 121 | TGATTTACGG GAAATGAGCC AACCAGGAAA CCCGTTTTTT |
| 161 | CTGACGTAAG GGTGCGCA |

As another example, an Eco2 STAT3 Retron Decoy ncRNA that includes a STAT3 binding site is shown below as SEQ ID NO: 26, where the STAT3 binding site is highlighted in bold with underlining.

| | |
|---|---|
| 1 | CACGCATGTA GGCAGATTTG TTGGTTGTGA ATCGCAACCA |
| 41 | GTGGCCTTAA TGGCAGGAGG AATCGCCTCC CTAAAATCGT |
| 81 | AAAGGGCATT TAGCTTCTAA ATGCCCTTTA CGATTCAGAG |
| 121 | CTATACGGCA GGTGTGCTGT GCGAAGGAGT GCCTGCATGC |
| 161 | GT |

Variations in transcription factor binding site sequences can occur amongst natural populations of retrons. Such binding sites with sequence variations can also be used as retron decoys. For example, any of the binding site and retron sequences described herein can have at least about 80-100% sequence identity to the sequences described herein, including any percent identity within this range, such as at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a nucleotide sequence described herein.

### Expression Systems

In certain embodiments, the retron ncRNA and RT gene are expressed in vivo from a vector within a cell. A “vector” is a composition of matter which can be used to deliver a nucleic acid of interest to the interior of a cell. The retron ncRNA and RT gene can be introduced into a cell with a single vector or in multiple separate vectors to produce msDNA in a host subject. Vectors typically include control elements operably linked to the retron sequences, which allow for the production of msDNA in vivo in the subject species. For example, the retron msr, msd, and RT gene can be operably linked to a promoter to allow expression of the retron reverse transcriptase and msDNA product. The transcription factor binding sites may be inserted in the msr gene or msd gene. Any eukaryotic, archeon, or prokaryotic cell, capable of being transfected with a vector comprising the engineered retron sequences, may be used to produce the msDNA. The ability of constructs to produce the msDNA along that include the transcription factor binding site(s) can be empirically determined.

In some embodiments, the engineered retron is produced by a vector system comprising one or more vectors. In the vector system, the msr, the msd, and the RT gene may be provided by the same vector (i.e., cis arrangement of all such retron elements), wherein the vector comprises a promoter operably linked to the msr and the msd. In some embodiments, the promoter is further operably linked to the RT gene. In other embodiments, the vector further comprises a second promoter operably linked to the RT gene. Alternatively, the RT gene may be provided by a second vector that does not include the msr and the msd (i.e., trans arrangement of msr-msd and RT). In yet other embodiments, the msr, the msd, and the RT gene are each provided by different vectors (i.e., trans arrangement of all retron elements). Numerous vectors are available including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid comprising an engineered retron sequence is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

In one embodiment, an expression vector for expressing an engineered retron, including the msr, msd, and RT gene comprises a promoter “operably linked” to a polynucleotide encoding the msr, msd, and RT gene. The phrase “operably linked” or “under transcriptional control” as used herein means that the promoter is in the correct location and orientation in relation to a polynucleotide to control the initiation of transcription by RNA polymerase and expression of the msr, msd, and RT gene. In another embodiment, an expression vector is used for expressing an engineered retron, including the msr and/or msd, operably linked to a promoter.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES).

Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20:102-110; Kobayashi et al., BioTechniques (1996) 21:399-402; and Mosser et al., BioTechniques (1997) 22: 150-161. A multitude of IRES sequences are known and include sequences derived from a wide variety of viruses, such as from leader sequences of picornaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25):15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17):7622-7635), vascular endothelial growth factor IRES (Baranick et al. (2008) Proc. Natl. Acad. Sci. U.S.A. 105(12):4733-4738, Stein et al. (1998) Mol. Cell. Biol. 18(6):3112-3119, Bert et al. (2006) RNA 12(6):1074-1083), and insulin-like growth factor 2 IRES (Pedersen et al. (2002) Biochem. J. 363(Pt 1):37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express a retron reverse transcriptase from an expression cassette.

Alternatively, a polynucleotide encoding a viral T2A peptide can be used to allow production of multiple protein products (e.g., Cas9, bacteriophage recombination proteins, retron reverse transcriptase) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLoS One 6(4):e18556, Trichas et al. (2008) BMC Biol. 6:40, Provost et al. (2007) Genesis 45(10):625-629, Furler et al. (2001) Gene Ther. 8(11):864-873; herein incorporated by reference in their entireties.

In certain embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (γ-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2):249-271; and Lundstrom (2003) Trends Biotechnol. 21(3):117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24):2516-2527). Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2):132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2^{nd} edition, 2007) and L. King, The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

In order to effect expression of engineered retron constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid comprising the engineered retron sequence may be positioned and expressed at different sites. In certain embodiments, the nucleic acid comprising the engineered retron sequence may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids comprising the engineered retron. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding an engineered retron of interest may also be transferred in a similar manner in vivo and express retron products.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposome may be complexed with a hemagglutinin virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising an engineered retron may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP:cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from a subject, the delivery of a nucleic acid into cells in vitro, and then the return of the modified cells back into the subject. This may involve the collection of a biological sample comprising cells from the subject. For example, blood can be obtained by venipuncture, and solid tissue samples can be obtained by surgical techniques according to methods available in the art.

Usually, but not always, the subject who receives the cells (i.e., the recipient) is also the subject from whom the cells are harvested or obtained, which provides the advantage that the donated cells are autologous. However, cells can be obtained from another subject (i.e., donor), a culture of cells from a donor, or from established cell culture lines. Cells may be obtained from the same or a different species than the subject to be treated, but preferably are of the same species, and more preferably of the same immunological profile as the subject. Such cells can be obtained, for example, from a biological sample comprising cells from a close relative or matched donor, then transfected with nucleic acids (e.g., comprising an engineered retron), and administered to a subject in need of genome modification, for example, for treatment of a disease or condition.

### Kits

Also provided are kits comprising engineered retron constructs as described herein. In some embodiments, the kit provides an engineered retron construct or an expression cassette/vector system comprising such a retron construct. In some embodiments, the engineered retron construct or a series of engineered retrons included in the kit, each have one or more transcription factor binding sites suitable for use in modulating gene expression. Other agents may also be included in the kit such as transfection agents, host cells, suitable media for culturing cells, buffers, and the like.

In the context of a kit, agents can be provided in liquid or sold form in any convenient packaging (e.g., vials, powder packs, etc.). The agents of a kit can be present in the same or separate containers. The agents may also be present in the same container. In addition to the above components, the subject kits may further include (in certain embodiments) instructions for generating engineered retrons, cells that can express engineered retrons, methods for using engineered retrons. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Therapeutic Methods

The engineered retrons, expression systems encoding the engineered retrons, and/or cells containing the engineered retrons or expression systems can be administered to a subject. Such a subject may suffer from a disease or condition or be suspected of suffering from a disease or condition. Symptoms of the disease or condition can be reduced by such administration. In some cases, progression of the disease or condition can be prevented or reduced by such administration. In some cases, the subject may be asymptomatic but be genetically pre-disposed to developing disease or condition.

Hence, described herein are methods of administering one or more engineered retrons, expression systems encoding the engineered retrons, and/or cells containing the engineered retrons or expression systems to a subject. The methods can provide prophylaxis, amelioration and/or therapy for a variety of diseases or conditions, including pain, ischemic diseases, allergic diseases, inflammatory diseases, autoimmune diseases, metastasis/infiltration of cancers, cachexia, vascular restenosis, acute coronary syndrome, brain ischemia, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), cystic fibrosis (CF), vascular restenosis which occurs after PTCA (percutaneous transluminal coronary angioplasty), PTA (percutaneous transluminal angioplasty), bypass surgery, organ transplantation, surgery of an organ (including those caused by using an artificial blood vessel, catheter or stent, or by vein grafting, and including those caused by a surgical treatment for arteriosclerosis obliterans, aneurysm, aorta dissection, acute coronary syndrome, brain ischemia, Marfan syndrome or plaque rupture), and combinations thereof.

The methods modulate (e.g., reduce) binding of transcription factors to endogenous regulatory sites that regulate expression of one or more genes to thereby reduce the onset, symptoms, or progression of the disease. For example, an engineered retron which contains a binding sequence for NF-κB acts as a decoy for NF-κB. Such an engineered retron can be used as an agent for prophylaxis, amelioration and/or therapy of any diseases caused by NF-κB, including ischemic diseases, allergic diseases, inflammatory diseases, autoimmune diseases and tumors. In another example, an engineered retron which contains a binding sequence for a KLF transcription factor can be used for preventing and/or treating conditions relating to KLF, such as pain in a subject. In some embodiments, the pain is a chronic pain, or neuropathic pain, or pain associated with inflammation. In certain embodiments, the pain is associated with central nervous system or visceral disorder. In specific embodiments the pain is neuropathic pain associated with inflammation.

The following Examples illustrate some of the experiments performed in the develop of the invention.

### Example 1

### Materials and Methods

This Example illustrates some of the materials methods used in developing the invention.

Synthesized gBlocks encoding human codon optimized Eco1 and Eco2 were cloned into a PiggyBac-integrating plasmid for doxycycline-inducible human protein expression (TetOn-3G promoter). Eco1 variants employed included the wild-type retron-Eco1 RT and ncRNA (pKDC.018) as well as an Eco1 ncRNA containing the STAT3 binding motif in the stem structure of the RT-DNA (pKDC.035). Eco2 variants used included the wild-type retron-Eco2 RT and ncRNA (pKDC.015) as well as an Eco2 ncRNA containing the STAT3 binding motif in the stem structure of the RT-DNA (pKDC.023).

Stable mammalian cell lines were created using the Lipofectamine 3000 transfection protocol (Invitrogen) and a PiggyBac transposase system. T25 flasks of 50-70% confluent HEK293T cells were transfected using 8.3 ug of retron expression plasmids (pKDC.015, pKDC.018, pKDC.019, pKDC.020, or pKDC.031) and 4.2 ug PiggyBac transposase plasmid (pCMV-hyPBase). Stable cell lines were selected with puromycin.

Ligated decoy oligonucleotides were generated as follows. Single-stranded DNA oligonucleotides containing the forward and reverse strand of the STAT3 decoy sequence separated by hexylene glycol linkers were formulated by Integrated DNA Technologies. The single-stranded decoys were resuspended at 800 μM and heated for 5 minutes at 55° C. A solution of 15 μL of 800 uM decoy, 2 μL T4 DNA ligase buffer, and 3 μL T4 DNA ligase buffer was incubated overnight at room temperature (NEB). The solution as then heated for 10 minutes at 65° C. and run on a 15% TBE-Urea gel to verify ligation (Invitrogen).

HEK293T and Cal33 cells were maintained in high-glucose DMEM with L-glutamine and 10% v/v heat-inactivated fetal bovine serum (Gibco). RetroDecoy expression in stable HEK293T cell lines was induced using 1 μg/mL doxycycline for 24 h at 37° C. in 6-well plates. Aliquots (1 ml) of induced and uninduced cell lines were collected for RNA extraction. Ligated oligonucleotide decoys were transfected into Cal33 cells using Lipofectamine 2000 (Invitrogen). Briefly, 50,000 Cal33 cells were seeded in 12-well plates. Twelve hours after plating, ligated STAT3 decoy and control oligonucleotides were mixed with OptiMEM media at 200 nM concentration at 200 ul volume and incubated 5 minutes. Lipofectamine 2000 (4 μl) was mixed with 196 μl of OptiMEM media and incubated 5 minutes. The Lipofectamine mixture was added to the DNA mixture (now 100 nM decoy or control oligonucleotide), mixed, and incubated 20 minutes at room temperature. After incubation, the maintenance media was aspirated from the Cal33 cells and 400 ul Lipofectamine/decoy oligonucleotide solution was added to the cells. After 24 hours of incubation at 37° C., the cells were collected for RNA extraction. RNA was extracted following manufacturer instructions using the RNeasy Mini kit (QIAGEN).

Reverse transcription-qPCR analysis of Myc expression levels was performed by comparing amplification from samples using two sets of primers. One set was used to amplify GAPDH mRNA (forward: ACCCACTCCTCCACCTTTGAC (SEQ ID NO: 27); reverse: TGTTGCTGTAGCCAAATTCGTT (SEQ ID NO: 28)) and the other was used to amplify Myc mRNA (forward: GGGTAGTGGAAAACCAGCAG (SEQ ID NO: 29); reverse: CAGCAGCTCGAATTTCTTCC (SEQ ID NO: 30)). Results were analyzed by first taking the difference in cycle threshold (CT) between the Myc and GAPDH primer set for each biological replicate. The ACT value for each biological replicate was subtracted from the ACT value of the control condition relating to that biological replicate (i.e., uninduced for retroDecoy experiments or sham transfection for oligonucleotide experiments). The fold change was calculated as 2^{-ΔΔCT}, where downregulation of Myc by decoys leads to fold change values of greater than 1.

### Example 2: RetroDecoys for STAT3, an Oncogenic Transcription Factor

This Example illustrates use of a retron decoy (RetroDecoy) to modulate (e.g., reduce) transcription mediated by the STAT3 transcription factor.

STAT3 is an oncogenic transcription factor that can induce Myc transcription. The targeted STAT3 binding motif was: CATTTCCCGTAAATC (SEQ ID NO: 31). The inventors created RetroDecoys for the oncogenic transcription factor STAT3, using modified ncRNAs from two retrons, Eco1 and Eco2. The Retron Decoys are retron reverse-transcribed DNA (RT-DNA) that include the STAT3 binding motif.

The Eco1 human-codon optimized RT sequence used is shown below as SEQ ID NO: 32.

| | |
|---|---|
| 1 | ATGAAATCTG CAGAGTATCT GAATACGTTC CGCCTTAGGA |
| 41 | ATTTGGGCCT CCCCGTGATG AACAATCTCC ACGATATGAG |
| 81 | CAAGGCGACT CGAATATCCG TGGAAACGCT GAGACTGCTC |
| 121 | ATCTATACAG CAGACTTTCG GTACAGGATC TACACGGTCG |
| 161 | AAAAGAAGGG GCCTGAGAAA CGCATGCGAA CAATTTATCA |
| 201 | ACCTAGCCGA GAGCTCAAGG CGTTGCAGGG CTGGGTTCTT |
| 241 | CGAAACATCC TTGACAAACT CTCATCATCA CCCTTTAGTA |
| 281 | TTGGGTTTGA AAAGCACCAA AGCATCCTTA ACAACGCGAC |
| 321 | GCCACACATA GGTGCCAATT TCATATTGAA CATCGACTTG |
| 361 | GAGGATTTTT TTCCGAGCCT CACAGCCAAT AAAGTGTTCG |
| 401 | GTGTTTTTCA CAGTCTTGGG TACAATCGCC TTATTAGTTC |
| 411 | CGTTCTTACC AAGATTTGTT GTTACAAGAA TCTCTTGCCC |
| 481 | CAGGGAGCAC CCAGCAGTCC GAAATTGGCG AATTTGATTT |
| 521 | GTTCCAAGCT CGATTATCGA ATACAAGGGT ACGCGGGCAG |
| 561 | CCGGGGACTC ATCTATACCC GCTACGCAGA CGATCTTACG |
| 601 | CTGTCTGCCC AATCAATGAA GAAGGTCGTA AAGGCGCGGG |
| 641 | ATTTCTTGTT TTCTATCATC CCGTCCGAGG GCTTGGTAAT |
| 681 | TAATTCCAAA AAGACTTGTA TCTCAGGACC ACGATCTCAG |
| 721 | CGAAAAGTGA CAGGACTCGT CATTTCTCAA GAAAAAGTCG |
| 761 | GTATAGGGAG AGAGAAGTAT AAGGAAATCC GCGCGAAGAT |
| 801 | CCACCACATA TTCTGTGGCA AGAGCAGCGA GATAGAACAC |
| 841 | GTCCGAGGCT GGTTGTCCTT CATACTGAGC GTGGACTCAA |
| 881 | AAAGCCACCG CCGGTTGATC ACCTATATTT CAAAACTGGA |
| 921 | AAAGAAATAT GGAAAGAACC CACTCAACAA AGCTAAAACA |
| 961 | TAG |

The Eco1 wild-type ncRNA sequence is from pKDC.018 and is shown below as SEQ ID NO: 33.

| | |
|---|---|
| 1 | ATGCGCACCC TTAGCGAGAG GTTTATCATT AAGGTCAACC |
| 41 | TCTGGATGTT GTTTCGGCAT CCTGCATTGA ATCTGAGTTA |
| 81 | CTGTCTGTTT TCCTTGTTGG AACGGAGAGC ATCGCCTGAT |
| 121 | GCTCTCCGAG CCAACCAGGA AACCCGTTAT TTCTGACGTA |
| 161 | AGGGTGCGCA |

The Eco1 STAT3 Retron Decoy ncRNA sequence from pKDC.035 and is shown below as SEQ ID NO:34.

| | |
|---|---|
| 1 | ATGCGCACCC TTAGCGAGAG GTTTATCATT AAGGTCAACC |
| 41 | TCTGGATGTT GTTTCGGCAT CCTGCATTGA ATCTGAGTTA |
| 81 | CTGTCTGTTT TCCTTGTTGG AACATTTCCC GTAAATCGCC |
| 121 | TGATTTACGG GAAATGAGCC AACCAGGAAA CCCGTTTTTT |
| 161 | CTGACGTAAG GGTGCGCA |

The Eco2 human-codon optimized RT sequence is shown below as SEQ ID NO: 35.

| | |
|---|---|
| 1 | ATGACAAAAA CTTCAAAGCT GGATGCGCTG CGGGCGGCTA |
| 41 | CTAGTAGGGA AGATTTGGCG AAGATTCTCG ACATAAAGTT |
| 81 | GGTGTTTCTG ACAAACGTGT TGTACCGCAT AGGATCCGAC |
| 121 | AACCAGTATA CGCAATTCAC AATACCCAAA AAGGGTAAAG |
| 161 | GTGTCCGCAC CATCAGCGCA CCAACGGACC GACTTAAGGA |
| 201 | TATACAGAGG AGGATTTGTG ATCTTCTTAG TGACTGTAGG |
| 241 | GATGAAATCT TTGCGATTAG GAAGATCTCT AATAATTACT |
| 281 | CATTCGGCTT CGAAAGAGGA AAATCAATTA TACTCAATGC |
| 321 | TTACAAGCAT CGAGGGAAGC AAATTATATT GAACATCGAC |
| 361 | CTTAAGGACT TCTTTGAGAG CTTTAACTTT GGGAGAGTCC |
| 401 | GGGGGTACTT TCTCTCCAAC CAGGACTTCT TGTTGAACCC |
| 441 | AGTIGTGGCA ACAACGTTGG CGAAGGCCGC CTGCTACAAC |
| 481 | GGGACTCTGC CTCAGGGGTC CCCATGTTCC CCTATTATAA |
| 521 | GTAACCTTAT CTGTAACATT ATGGACATGC GGCTCGCAAA |
| 561 | GCTCGCCAAG AAGTACGGCT GCACTTATAG TCGATATGCG |
| 601 | GATGACATTA CGATCAGCAC CAATAAAAAT ACCTTCCCGT |
| 641 | TGGAGATGGC GACTGTGCAG CCTGAAGGGG TTGTGCTGGG |
| 681 | CAAAGTGCTC GTAAAGGAGA TTGAAAATTC AGGTTTCGAG |
| 721 | ATTAACGATT CTAAGACTAG ATTGACCTAC AAAACAAGTA |
| 761 | GGCAAGAAGT CACCGGGCTG ACGGTTAATC GGATTGTAAA |
| 801 | CATTGATCGG TGCTACTACA AAAAGACGAG GGCGCTGGCT |
| 841 | CACGCATTGT ATCGGACAGG AGAATATAAG GTCCCAGACG |
| 881 | AGAACGGTGT TCTGGTATCT GGAGGGCTTG ACAAGTTGGA |
| 921 | GGGTATGTTT GGGTTTATCG ACCAGGTGGA TAAATTCAAC |
| 961 | AACATTAAAA AAAAGTTGAA TAAGCAACCC GACAGATATG |
| 1001 | TTCTGACAAA TGCCACTTTG CACGGATTTA AGCTCAAATT |
| 1041 | GAACGCCAGG GAGAAAGCCT ATAGCAAATT CATCTACTAC |
| 1081 | AAATTCTTCC ACGGTAATAC TTGTCCCACG ATCATAACAG |
| 1121 | AGGGTAAGAC GGATAGGATT TACCTTAAAG CTGCCCTCCA |
| 1161 | TAGCCTCGAG ACAAGTTATC CTGAACTGTT TCGGGAGAAA |
| 1201 | ACAGATAGTA AGAAGAAGGA GATAAATCTG AATATTTTTA |
| 1241 | AAAGCAATGA GAAGACCAAG TATTTCCTGG ATCTCAGCGG |
| 1281 | CGGCACAGCA GACCTCAAGA AATTCGTGGA ACGCTACAAA |
| 1321 | AATAACTACG CTTCCTATTA CGGCAGCGTA CCGAAACAAC |
| 1361 | CGGTGATAAT GGTGCTTGAT AACGACACAG GCCCGTCAGA |
| 1401 | CCTGTTGAAC TTTTTGAGAA ACAAAGTTAA GAGTTGTCCA |
| 1441 | GATGATGTAA CAGAAATGCG CAAGATGAAG TACATACATG |
| 1481 | TGTTTTACAA TCTGTACATA GTTCTGACTC CCCTGTCTCC |
| 1521 | ATCTGGAGAG CAAACGTCTA TGGAGGACCT CTTTCCTAAA |
| 1561 | GATATATTGG ACATTAAGAT AGATGGCAAG AAATTCAATA |
| 1601 | AAAACAATGA CGGTGACTCC AAAACAGAGT ATGGGAAGCA |
| 1001 | CATATTCTCA ATGCGCGTTG TACGAGATAA AAAGAGGAAG |
| 1001 | ATAGATTTCA AGGCATTTTG CTGTATCTTC GATGCTATTA |
| 1001 | AGGATATTAA AGAACATTAC AAACTGATGT TGAATTCCTA |
| 1001 | G |

The Eco2 wild type ncRNA sequence is from pKDC.015 and is shown below as SEQ ID NO: 36.

| | |
|---|---|
| 1 | CACGCATGTA GGCAGATTTG TTGGTTGTGA ATCGCAACCA |
| 41 | GTGGCCTTAA TGGCAGGAGG AATCGCCTCC CTAAAATCCT |
| 81 | TGATTCAGAG CTATACGGCA GGTGTGCTGT GCGAAGGAGT |
| 121 | GCCTGCATGC GT |

The Eco2 STAT3 Retron Decoy ncRNA sequence is from pKDC.023 and is shown below as SEQ ID NO: 37.

| | |
|---|---|
| 1 | CACGCATGTA GGCAGATTTG TTGGTTGTGA ATCGCAACCA |
| 41 | GTGGCCTTAA TGGCAGGAGG AATCGCCTCC CTAAAATCGT |
| 81 | AAAGGGCATT TAGCTTCTAA ATGCCCTTTA CGATTCAGAG |
| 121 | CTATACGGCA GGTGTGCTGT GCGAAGGAGT GCCTGCATGC |
| 161 | GT |

A STAT3 decoy oligonucleotide was also generated that had the following single-stranded sequence: 5′Phosphate-GTAAATC-hexylene glycol linker-GATTTACGGGAAATG (SEQ ID NO: 38)-hexylene glycol linker-CATTTCCC). A STAT3 mutant control oligonucleotide was used with the following single-stranded sequence: 5′Phosphate-TTAAATC-hexylene glycol linker-GATTTAAGGGAAATG (SEQ ID NO: 38)-hexylene glycol linker-CATTTCCC.

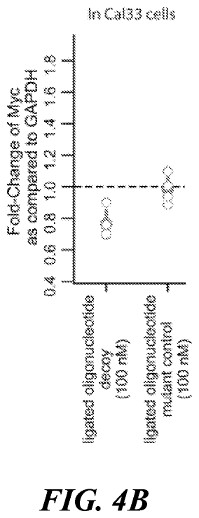

The Retron Decoys were expressed in cells from an integrated transgene via a small molecule induced promoter. As shown in FIG. 4A, the Eco1 STAT3 Retron Decoy ncRNA and the Eco2 STAT3 Retron Decoy ncRNA significantly downregulate transcription of the STAT3 target Myc, compared to matched wild type retron controls that did not contain the STAT3 binding motif.

The ligated decoy oligonucleotides with 5′ and 3′ ends protected through hexylene glycol linkers (100 nM) also downregulated Myc transcription when introduced to Cal33 tongue squamous cell carcinoma cells (FIG. 4B). However, such oligonucleotide decoys cannot be synthesized in vivo and cannot be targeted to particular cell types in vivo.

### REFERENCES

1 Inouye, M. & Inouye, S. msDNA and bacterial reverse transcriptase. Annual review of microbiology 45, 163-186, doi:10.1146/annurev.mi.45.100191.001115 (1991).
2 Farzadfard, F. & Lu, T. K. Synthetic biology. Genomically encoded analog memory with precise in vivo DNA writing in living cell populations. Science 346, 1256272, doi:10.1126/science.1256272 (2014).
3 Sharon, E. et al. Functional Genetic Variants Revealed by Massively Parallel Precise Genome Editing. Cell 175, 544-557.e516, doi:10.1016/j.cell.2018.08.057 (2018).
4 Dhundale, A. R., Furuichi, T., Inouye, S. & Inouye, M. Distribution of multicopy single-stranded DNA among myxobacteria and related species. Journal of bacteriology 164, 914-917 (1985).
5 Lampson, B. C. et al. Reverse transcriptase in a clinical strain of Escherichia coli: production of branched RNA-linked msDNA. Science 243, 1033-1038 (1989).
6 Inouye, K., Tanimoto, S., Kamimoto, M., Shimamoto, T. & Shimamoto, T. Two novel retron elements are replaced with retron-Vc95 in Vibrio cholerae. Microbiology and immunology 55, 510-513, doi:10.1111/j.1348-0421.2011.00342.x (2011).
7 Lim, D. Structure and biosynthesis of unbranched multicopy single-stranded DNA by reverse transcriptase in a clinical Escherichia coli isolate. Molecular microbiology 6, 3531-3542 (1992).
8 Shipman, S. L., Nivala, J., Macklis, J. D. & Church, G. CRISPR-Cas encoding of a digital movie into the genomes of a population of living bacteria. Nature, doi:10.1038/nature23017 (2017).
9 Shipman, S. L., Nivala, J., Macklis, J. D. & Church, G. M. Molecular recordings by directed CRISPR spacer acquisition. Science, doi:10.1126/science.aaf1175 (2016).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. An engineered retron comprising a transcription factor binding site within the retron msd region.
2. The engineered retron of statement 1, wherein the transcription factor binding site binds a AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor, or a combination of such transcription factors.
3. The engineered retron of statement 1 or 2, wherein retron does not include a segment encoding an accessory protein.
4. The engineered retron of any one of statements 1-3, wherein the retron is an engineered prokaryotic retron.
5. The engineered retron of any one of statements 1-4, wherein engineered retron is a modified Escherichia coli retron (e.g., Ec67, Ec73, EC83, EC86, EC107), myxobacteria retron (e.g., Mx65, Mx162), Salmonella enterica retron (e.g., St85, Se72), Vibrio cholerae retron (e.g., Vc81, Vc95, Vc137), Stigmatella aurantica retron (e.g., Sa163), Nannocystis exedens retron (Ne160, Ne144), Vibrio parahaemolyticus retron (e.g., Vp96), Proteus mirabilis retron (e.g., Pmi1), Klebsiella pneumoniae retron (e.g., Kpn1), Flexibacter elegans retron (e.g., Fei1), Corallococcus coralloides retron (e.g., Coo1), Cystobacter ferrugineus retron (e.g., Cfe1), Melittangium lichenicola retron (e.g., Mli1), Chondromyces apiculatus retron (e.g., Cap1), Sorangium cellulosum retron (e.g., Sce1), or combinations thereof.
6. The engineered retron of any one of statements 1-5, wherein engineered retron is a modified Eco1 or Eco2 retron.
7. An engineered retron msd DNA.
8. The engineered retron msd DNA of statement 7, wherein the transcription factor binding site binds a AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor, or a combination of such transcription factors.
9. The engineered retron msd DNA of statement 7 or 8, wherein retron does not include a segment encoding an accessory protein.
10. The engineered retron of msd DNA any one of statements 7-9, wherein the retron is an engineered prokaryotic retron.
11. The engineered retron msd DNA of any one of statements 7-10, wherein engineered retron is a modified Escherichia coli retron (e.g., Ec67, Ec73, EC83, EC86, EC107), myxobacteria retron (e.g., Mx65, Mx162), Salmonella enterica retron (e.g., St85, Se72), Vibrio cholerae retron (e.g., Vc81, Vc95, Vc137), Stigmatella aurantica retron (e.g., Sa163), Nannocystis exedens retron (Ne160, Ne144), Vibrio parahaemolyticus retron (e.g., Vp96), Proteus mirabilis retron (e.g., Pmi1), Klebsiella pneumoniae retron (e.g., Kpn1), Flexibacter elegans retron (e.g., Fei1), Corallococcus coralloides retron (e.g., Coo1), Cystobacter ferrugineus retron (e.g., Cfe1), Melittangium lichenicola retron (e.g., Mli1), Chondromyces apiculatus retron (e.g., Cap1), Sorangium cellulosum retron (e.g., Sce1), or combinations thereof.
12. The engineered retron msd DNA of any one of statements 7-11, wherein engineered retron is a modified Eco1 or Eco2 retron.
13. A composition comprising the engineered retron or the engineered retron msd DNA of any one of statements 1-12.
14. A cell comprising the engineered retron or the engineered retron msd DNA of any one of statements 1-12, or the composition of statement 13.
15. A method comprising administering the engineered retron or the engineered retron msd DNA of any one of statements 1-12, or the composition of statement 13, or the cell of statement 14 to a subject.
16. The method of statement 15, wherein the cell is autologous to the subject.
17. The method of statement 15 or 16, wherein the subject suffers from a disease or condition, or is suspected of suffering from a disease or condition.
18. The method of statement 17, wherein the disease or condition is related to or exacerbated by expression of at least one AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor.
19. The method of statement 17 or 18, wherein the disease or condition is pain, ischemic diseases, allergic diseases, inflammatory diseases, autoimmune diseases, metastasis/infiltration of cancers, cachexia, vascular restenosis, acute coronary syndrome, brain ischemia, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), cystic fibrosis (CF), vascular restenosis which occurs after PTCA (percutaneous transluminal coronary angioplasty), PTA (percutaneous transluminal angioplasty), bypass surgery, organ transplantation, surgery of an organ (including those caused by using an artificial blood vessel, catheter or stent, or by vein grafting, and including those caused by a surgical treatment for arteriosclerosis obliterans, aneurysm, aorta dissection, acute coronary syndrome, brain ischemia, Marfan syndrome or plaque rupture), or combinations thereof.
20. An expression cassette or expression vector comprising a promoter operably linked to a nucleic acid segment encoding at least one retron non-coding RNA, wherein an msd DNA reverse transcribed from each retron non-coding RNA comprises at least one transcription factor binding site.
21. The expression cassette or expression vector of statement 20, wherein the transcription factor binding site binds a AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor, or a combination of such transcription factors.
22. The expression cassette or expression vector of statement 20 or 21, wherein retron does not include a segment encoding an accessory protein.
23. The expression cassette or expression vector of statement 20, 21 or 22, wherein the retron is an engineered prokaryotic retron.
24. The expression cassette or expression vector of any one of statements 20-23, wherein engineered retron is a modified Escherichia coli retron (e.g., Ec67, Ec73, EC83, EC86, EC107), myxobacteria retron (e.g., Mx65, Mx162), Salmonella enterica retron (e.g., St85, Se72), Vibrio cholerae retron (e.g., Vc81, Vc95, Vc137), Stigmatella aurantica retron (e.g., Sa163), Nannocystis exedens retron (Ne160, Ne144), Vibrio parahaemolyticus retron (e.g., Vp96), Proteus mirabilis retron (e.g., Pmi1), Klebsiella pneumoniae retron (e.g., Kpn1), Flexibacter elegans retron (e.g., Fei1), Corallococcus coralloides retron (e.g., Coo1), Cystobacter ferrugineus retron (e.g., Cfe1), Melittangium lichenicola retron (e.g., Mli1), Chondromyces apiculatus retron (e.g., Cap1), Sorangium cellulosum retron (e.g., Sce1), or combinations thereof.
25. The expression cassette or expression vector of any one of statements 20-24, wherein engineered retron is a modified Eco1 or Eco2 retron.
26. A composition comprising the expression cassette or expression vector of any one of statements 20-25.
27. A cell comprising the expression cassette or expression vector of any one of statements 20-25.
28. A method comprising administering: the expression cassette or expression vector of any one of statements 20-25, the composition of statement 26, or the cell of statement 27 to a subject.
29. The method of statement 28, wherein the cell is autologous to the subject.
30. The method of statement 28 or 29, wherein the subject suffers from a disease or condition, or is suspected of suffering from a disease or condition.
31. The method of statement 30, wherein the disease or condition is related or exacerbated by expression of at least one AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, or TCF21 transcription factor.
32. The method of statement 30 or 31, wherein the disease or condition is pain, ischemic diseases, allergic diseases, inflammatory diseases, autoimmune diseases, metastasis/infiltration of cancers, cachexia, vascular restenosis, acute coronary syndrome, brain ischemia, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), cystic fibrosis (CF), vascular restenosis which occurs after PTCA (percutaneous transluminal coronary angioplasty), PTA (percutaneous transluminal angioplasty), bypass surgery, organ transplantation, surgery of an organ (including those caused by using an artificial blood vessel, catheter or stent, or by vein grafting, and including those caused by a surgical treatment for arteriosclerosis obliterans, aneurysm, aorta dissection, acute coronary syndrome, brain ischemia, Marfan syndrome or plaque rupture), or combinations thereof.
33. A kit comprising at least one engineered retron comprising a transcription factor binding site within the retron msd region, at least one an expression cassette/vector system encoding an engineered retron comprising a transcription factor binding site within the retron msd region, or a combination thereof.
34. The kit of statement 33, further comprising instructions for using at least one engineered retron or at least one expression cassette/vector system encoding an engineered retron construct.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. An engineered retron comprising a transcription factor binding site within the retron msd region.

2. The engineered retron of claim 1, wherein the transcription factor binding site binds a AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor, or a combination of such transcription factors.

3. The engineered retron of claim 1, wherein retron does not include a segment encoding an accessory protein.

4. The engineered retron of claim 1, wherein the retron is an engineered prokaryotic retron.

5. The engineered retron of claim 1, wherein engineered retron is a modified Escherichia coli retron (e.g., Ec67, Ec73, EC83, EC86, EC107), myxobacteria retron (e.g., Mx65, Mx162), Salmonella enterica retron (e.g., St85, Se72), Vibrio cholerae retron (e.g., Vc81, Vc95, Vc137), Stigmatella aurantica retron (e.g., Sa163), Nannocystis exedens retron (Ne160, Ne144), Vibrio parahaemolyticus retron (e.g., Vp96), Proteus mirabilis retron (e.g., Pmi1), Klebsiella pneumoniae retron (e.g., Kpn1), Flexibacter elegans retron (e.g., Fei1), Corallococcus coralloides retron (e.g., Coo1), Cystobacter ferrugineus retron (e.g., Cfe1), Melittangium lichenicola retron (e.g., Mli1), Chondromyces apiculatus retron (e.g., Cap1), Sorangium cellulosum retron (e.g., Sce1), or combinations thereof.

6. The engineered retron of claim 1, wherein engineered retron is a modified Eco1 or Eco2 retron.

7. (canceled)

8. (canceled)

9. (canceled)

10. (canceled)

11. (canceled)

12. A composition comprising the engineered retron of claim 1.

13. A cell comprising the engineered retron of claim 1.

14. A method comprising administering the engineered retron of claim 1 to a subject.

15. (canceled)

16. The method of claim 15, wherein the subject suffers from a disease or condition, or is suspected of suffering from a disease or condition.

17. The method of claim 16, wherein the disease or condition is related to or exacerbated by expression of at least one AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor.

18. The method of claim 17, wherein the disease or condition is pain, ischemic diseases, allergic diseases, inflammatory diseases, autoimmune diseases, metastasis/infiltration of cancers, cachexia, vascular restenosis, acute coronary syndrome, brain ischemia, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), cystic fibrosis (CF), vascular restenosis which occurs after PTCA (percutaneous transluminal coronary angioplasty), PTA (percutaneous transluminal angioplasty), bypass surgery, organ transplantation, surgery of an organ (including those caused by using an artificial blood vessel, catheter or stent, or by vein grafting, and including those caused by a surgical treatment for arteriosclerosis obliterans, aneurysm, aorta dissection, acute coronary syndrome, brain ischemia, Marfan syndrome or plaque rupture), or combinations thereof.

19. An expression cassette or expression vector comprising a promoter operably linked to a nucleic acid segment encoding at least one retron non-coding RNA, wherein an msd DNA reverse transcribed from each retron non-coding RNA comprises at least one transcription factor binding site.

20. The expression cassette or expression vector of claim 20, wherein the transcription factor binding site binds a AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, TCF21 transcription factor, or a combination of such transcription factors.

21. The expression cassette or expression vector of claim 20, wherein retron does not include a segment encoding an accessory protein.

22. The expression cassette or expression vector of claim 20, wherein the retron is an engineered prokaryotic retron.

23. The expression cassette or expression vector of claim 20, wherein engineered retron is a modified Escherichia coli retron (e.g., Ec67, Ec73, EC83, EC86, EC107), myxobacteria retron (e.g., Mx65, Mx162), Salmonella enterica retron (e.g., St85, Se72), Vibrio cholerae retron (e.g., Vc81, Vc95, Vc137), Stigmatella aurantica retron (e.g., Sa163), Nannocystis exedens retron (Ne160, Ne144), Vibrio parahaemolyticus retron (e.g., Vp96), Proteus mirabilis retron (e.g., Pmi1), Klebsiella pneumoniae retron (e.g., Kpn1), Flexibacter elegans retron (e.g., Fei1), Corallococcus coralloides retron (e.g., Coo1), Cystobacter ferrugineus retron (e.g., Cfe1), Melittangium lichenicola retron (e.g., Mli1), Chondromyces apiculatus retron (e.g., Cap1), Sorangium cellulosum retron (e.g., Sce1), or combinations thereof.

24. The expression cassette or expression vector of claim 20, wherein engineered retron is a modified Eco1 or Eco2 retron.

25. A composition comprising the expression cassette or expression vector of claim 20.

26. A cell comprising the expression cassette or expression vector of claim 20.

27. A method comprising administering: the expression cassette or expression vector of claim 20 to a subject.

28. The method of claim 28, wherein the cell is autologous to the subject.

29. The method of claim 28, wherein the subject suffers from a disease or condition, or is suspected of suffering from a disease or condition.

30. The method of claim 30, wherein the disease or condition is related or exacerbated by expression of at least one AP-1 family (JunB), AP-1, ATF family, CBFb, CBFb-SMMHC, C/EBP family, E2F, ELKF, Erg/Flil, Ets, Forkhead/Ets, FOG1, FOSL2, FOXO family, GABP, GABPa, GATA family, GATA-2, GATA-3, GFI1b, glucocorticoid receptor, KLF1, KLF2, KLF3, KLF4, KLF5, KLF6, KLF7, KLF8, KLF9, KLF10, KLF11, KLF12, KLF13, KLF14, KLF15, KLF16, KLF17, LMO2, MEIS2, MLL-AF9, Myc, MZF1-A, NANOG, NF-κB, NKX2-5, OCT4, p53, PML-RARa, RUNX1, SIX1, Sp family, STAT1, STAT3, STAT6, SOX2, Tal1/Scl/Lyl1, TBX5, or TCF21 transcription factor.

31. The method of claim 30, wherein the disease or condition is pain, ischemic diseases, allergic diseases, inflammatory diseases, autoimmune diseases, metastasis/infiltration of cancers, cachexia, vascular restenosis, acute coronary syndrome, brain ischemia, myocardial infarction, reperfusion hindrance of ischemic diseases, atopic dermatitis, psoriasis vulgaris, contact dermatitis, keloid, decubital ulcer, ulcerative colitis, Crohn's disease, nephropathy, glomerulosclerosis, albuminuria, nephritis, renal failure, rheumatoid arthritis, osteoarthritis, asthma, chronic obstructive pulmonary disease (COPD), cystic fibrosis (CF), vascular restenosis which occurs after PTCA (percutaneous transluminal coronary angioplasty), PTA (percutaneous transluminal angioplasty), bypass surgery, organ transplantation, surgery of an organ (including those caused by using an artificial blood vessel, catheter or stent, or by vein grafting, and including those caused by a surgical treatment for arteriosclerosis obliterans, aneurysm, aorta dissection, acute coronary syndrome, brain ischemia, Marfan syndrome or plaque rupture), or combinations thereof.

32. (canceled)

33. (canceled)


SEQUENCE LISTING (43 sequences)

SEQ ID NO: 1 - DNA, 170 bp, Escherichia coli
  source 1..170: mol_type = other DNA
atgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttatttctgacgtaagggtgcgca

SEQ ID NO: 2 - DNA, 963 bp, Escherichia coli
  source 1..963: mol_type = other DNA
atgaaatctgcagagtatctgaatacgttccgccttaggaatttgggcctccccgtgatgaacaatctccacgatatgagcaaggcgactcgaatatccgtggaaacgctgagactgctcatctatacagcagactttcggtacaggatctacacggtcgaaaagaaggggcctgagaaacgcatgcgaacaatttatcaacctagccgagagctcaaggcgttgcagggctgggttcttcgaaacatccttgacaaactctcatcatcaccctttagtattgggtttgaaaagcaccaaagcatccttaacaacgcgacgccacacataggtgccaatttcatattgaacatcgacttggaggatttttttccgagcctcacagccaataaagtgttcggtgtttttcacagtcttgggtacaatcgccttattagttccgttcttaccaagatttgttgttacaagaatctcttgccccagggagcacccagcagtccgaaattggcgaatttgatttgttccaagctcgattatcgaatacaagggtacgcgggcagccggggactcatctatacccgctacgcagacgatcttacgctgtctgcccaatcaatgaagaaggtcgtaaaggcgcgggatttcttgttttctatcatcccgtccgagggcttggtaattaattccaaaaagacttgtatctcaggaccacgatctcagcgaaaagtgacaggactcgtcatttctcaagaaaaagtcggtatagggagagagaagtataaggaaatccgcgcgaagatccaccacatattctgtggcaagagcagcgagatagaacacgtccgaggctggttgtccttcatactgagcgtggactcaaaaagccaccgccggttgatcacctatatttcaaaactggaaaagaaatatggaaagaacccactcaacaaagctaaaacatag

SEQ ID NO: 3 - DNA, 1761 bp, Escherichia coli
  source 1..1761: mol_type = other DNA
atgacaaaaacttcaaagctggatgcgctgcgggcggctactagtagggaagatttggcgaagattctcgacataaagttggtgtttctgacaaacgtgttgtaccgcataggatccgacaaccagtatacgcaattcacaatacccaaaaagggtaaaggtgtccgcaccatcagcgcaccaacggaccgacttaaggatatacagaggaggatttgtgatcttcttagtgactgtagggatgaaatctttgcgattaggaagatctctaataattactcattcggcttcgaaagaggaaaatcaattatactcaatgcttacaagcatcgagggaagcaaattatattgaacatcgaccttaaggacttctttgagagctttaactttgggagagtccgggggtactttctctccaaccaggacttcttgttgaacccagttgtggcaacaacgttggcgaaggccgcctgctacaacgggactctgcctcaggggtccccatgttcccctattataagtaaccttatctgtaacattatggacatgcggctcgcaaagctcgccaagaagtacggctgcacttatagtcgatatgcggatgacattacgatcagcaccaataaaaataccttcccgttggagatggcgactgtgcagcctgaaggggttgtgctgggcaaagtgctcgtaaaggagattgaaaattcaggtttcgagattaacgattctaagactagattgacctacaaaacaagtaggcaagaagtcaccgggctgacggttaatcggattgtaaacattgatcggtgctactacaaaaagacgagggcgctggctcacgcattgtatcggacaggagaatataaggtcccagacgagaacggtgttctggtatctggagggcttgacaagttggagggtatgtttgggtttatcgaccaggtggataaattcaacaacattaaaaaaaagttgaataagcaacccgacagatatgttctgacaaatgccactttgcacggatttaagctcaaattgaacgccagggagaaagcctatagcaaattcatctactacaaattcttccacggtaatacttgtcccacgatcataacagagggtaagacggataggatttaccttaaagctgccctccatagcctcgagacaagttatcctgaactgtttcgggagaaaacagatagtaagaagaaggagataaatctgaatatttttaaaagcaatgagaagaccaagtatttcctggatctcagcggcggcacagcagacctcaagaaattcgtggaacgctacaaaaataactacgcttcctattacggcagcgtaccgaaacaaccggtgataatggtgcttgataacgacacaggcccgtcagacctgttgaactttttgagaaacaaagttaagagttgtccagatgatgtaacagaaatgcgcaagatgaagtacatacatgtgttttacaatctgtacatagttctgactcccctgtctccatctggagagcaaacgtctatggaggacctctttcctaaagatatattggacattaagatagatggcaagaaattcaataaaaacaatgacggtgactccaaaacagagtatgggaagcacatattctcaatgcgcgttgtacgagataaaaagaggaagatagatttcaaggcattttgctgtatcttcgatgctattaaggatattaaagaacattacaaactgatgttgaattcctag

SEQ ID NO: 4 - DNA, 132 bp, Escherichia coli
  source 1..132: mol_type = other DNA
cacgcatgtaggcagatttgttggttgtgaatcgcaaccagtggccttaatggcaggaggaatcgcctccctaaaatccttgattcagagctatacggcaggtgtgctgtgcgaaggagtgcctgcatgcgt

SEQ ID NO: 5 - DNA, 15 bp, synthetic construct
  source 1..15: mol_type = other DNA
catttcccgtaaatc

SEQ ID NO: 6 - DNA, 20 bp, synthetic construct
  source 1..20: mol_type = other DNA
ccttgaggggatttcccccc

SEQ ID NO: 7 - DNA, 15 bp, synthetic construct
  source 1..15: mol_type = other DNA
ggtacannntgtnct

SEQ ID NO: 8 - DNA, 37 bp, synthetic construct
  source 1..37: mol_type = other DNA
gatcctttgcctccttcgatcctttgcctccttcaag

SEQ ID NO: 9 - DNA, 29 bp, synthetic construct
  source 1..29: mol_type = other DNA
ggtgtttgggagagctttgggaggatacg

SEQ ID NO: 10 - DNA, 16 bp, synthetic construct
  source 1..16: mol_type = other DNA
cggacgcgcattangc

SEQ ID NO: 11 - DNA, 22 bp, synthetic construct
  source 1..22: mol_type = other DNA
tcttctcacacctttgaagtgg

SEQ ID NO: 12 - DNA, 11 bp, synthetic construct
  source 1..11: mol_type = other DNA
cataacaggaa

SEQ ID NO: 13 - DNA, 11 bp, synthetic construct
  source 1..11: mol_type = other DNA
ggggcggggcg

SEQ ID NO: 14 - DNA, 16 bp, synthetic construct
  source 1..16: mol_type = other DNA
gggcggtcccgggcgg

SEQ ID NO: 15 - DNA, 11 bp, synthetic construct
  source 1..11: mol_type = other DNA
ggggcggggcg

SEQ ID NO: 16 - DNA, 11 bp, synthetic construct
  source 1..11: mol_type = other DNA
ggggcggggcg

SEQ ID NO: 17 - DNA, 11 bp, synthetic construct
  source 1..11: mol_type = other DNA
cttcctagaaa

SEQ ID NO: 18 - DNA, 15 bp, synthetic construct
  source 1..15: mol_type = other DNA
agaacagaatgttct

SEQ ID NO: 19 - DNA, 12 bp, synthetic construct
  source 1..12: mol_type = other DNA
acaggaagatnn

SEQ ID NO: 20 - skipped

SEQ ID NO: 21 - DNA, 11 bp, synthetic construct
  source 1..11: mol_type = other DNA
tgcagctgctg

SEQ ID NO: 22-23 - skipped (2 entries)

SEQ ID NO: 24 - DNA, 14 bp, synthetic construct
  source 1..14: mol_type = other DNA
cgggngagggggaa

SEQ ID NO: 25 - DNA, 178 bp, Escherichia coli
  source 1..178: mol_type = other DNA
atgcgcacccttagcgagaggtttatcattaaggtcaacctctggatgttgtttcggcatcctgcattgaatctgagttactgtctgttttccttgttggaacatttcccgtaaatcgcctgatttacgggaaatgagccaaccaggaaacccgttttttctgacgtaagggtgcgca

SEQ ID NO: 26 - DNA, 162 bp, Escherichia coli
  source 1..162: mol_type = other DNA
cacgcatgtaggcagatttgttggttgtgaatcgcaaccagtggccttaatggcaggaggaatcgcctccctaaaatcgtaaagggcatttagcttctaaatgccctttacgattcagagctatacggcaggtgtgctgtgcgaaggagtgcctgcatgcgt

SEQ ID NO: 27 - DNA, 21 bp, synthetic construct
  source 1..21: mol_type = other DNA
acccactcctccacctttgac

SEQ ID NO: 28 - DNA, 22 bp, synthetic construct
  source 1..22: mol_type = other DNA
tgttgctgtagccaaattcgtt

SEQ ID NO: 29 - DNA, 20 bp, synthetic construct
  source 1..20: mol_type = other DNA
gggtagtggaaaaccagcag

SEQ ID NO: 30 - DNA, 20 bp, synthetic construct
  source 1..20: mol_type = other DNA
cagcagctcgaatttcttcc

SEQ ID NO: 31 - DNA, 15 bp, synthetic construct
  source 1..15: mol_type = other DNA
catttcccgtaaatc

SEQ ID NO: 32 - DNA, 963 bp, synthetic construct; identical to SEQ ID NO: 2
  source 1..963: mol_type = other DNA

SEQ ID NO: 33 - DNA, 170 bp, Escherichia coli; identical to SEQ ID NO: 1
  source 1..170: mol_type = other DNA

SEQ ID NO: 34 - DNA, 178 bp, synthetic construct; identical to SEQ ID NO: 25
  source 1..178: mol_type = other DNA

SEQ ID NO: 35 - DNA, 1761 bp, synthetic construct; identical to SEQ ID NO: 3
  source 1..1761: mol_type = other DNA

SEQ ID NO: 36 - DNA, 132 bp, Escherichia coli; identical to SEQ ID NO: 4
  source 1..132: mol_type = other DNA

SEQ ID NO: 37 - DNA, 162 bp, synthetic construct; identical to SEQ ID NO: 26
  source 1..162: mol_type = other DNA

SEQ ID NO: 38 - DNA, 15 bp, synthetic construct
  source 1..15: mol_type = other DNA
gatttaagggaaatg

SEQ ID NO: 39 - DNA, 10 bp, synthetic construct
  source 1..10: mol_type = other DNA
aaatcacagc

SEQ ID NO: 40 - DNA, 12 bp, synthetic construct
  source 1..12: mol_type = other DNA
ccctgcgcaggg

SEQ ID NO: 41 - DNA, 10 bp, synthetic construct
  source 1..10: mol_type = other DNA
cacttcctgt

SEQ ID NO: 42 - DNA, 18 bp, synthetic construct
  source 1..18: mol_type = other DNA
gaggtcaaaaggtcaatg

SEQ ID NO: 43 - DNA, 17 bp, synthetic construct
  source 1..17: mol_type = other DNA
ngntcannnnnaggtca